(19) United States
(12) Patent Application Publication
(10) Pub. No.: US 2023/0213601 A1
(43) Pub. Date: Jul. 6, 2023

(54) Method For Detecting The Presence Of Abnormal Tissue

(71) Applicant: SOFTSYSTEM SPOLKA Z OGRANICZONA ODPOWIEDZIALNOSCIA (PL), Rzeszow (PL)

(72) Inventors: Michal Madera, Niechobrz (PL); Marta Micek, Rzeszow (PL); Justyna Surowka, Krasne (PL); Dorota Bartusik-Aebisher, Bedziemysl (PL); Dawid Aebisher, Bedziemysl (PL); Ewa Kaznowska, Rzeszow (PL); Jacek Tabarkiewicz, Kraczkowa (PL)

(21) Appl. No.: 17/718,409

(22) Filed: Apr. 12, 2022

(30) Foreign Application Priority Data
Dec. 30, 2021 (PL) P.440045

(51) Int. Cl. G01R 33/50; G01N 24/08; G16H 10/20

(52) U.S. Cl. CPC G01R 33/50; G01N 24/08; G16H 10/20

(57) ABSTRACT

A computer implemented method is usable to detect the presence of abnormal tissue through analysis of magnetic resonance relaxation times T1 and T2. The relaxation times T1 and T2 are determined from a data set obtained from a magnetic resonance apparatus. The method includes: loading the data set from at least one tissue into a computing device; determining a region of interest; determining an average value of the free induction decay signal within the region of interest on each of the scans separately; detecting scans with outlier data in each data series; and, if a scan with outlier data is detected, identifying the scan in the data series; determining the relaxation time within the region of interest based on scans from the corresponding data series that are not identified as having outlier data; classifying the tissue as normal or abnormal based on predefined values, which are determined depending on the type of tissue analyzed.

### TECHNICAL FIELD

Exemplary arrangements relate to the detection and analysis of abnormal tissue. Specific exemplary arrangements relate to the detection of cancerous lesions within mammary gland tissue through the evaluation of magnetic resonance imaging nuclear relaxation times. Further exemplary arrangements relate to methods for making diagnostic determinations and evaluations based on magnetic resonance imaging nuclear relaxation time data.

### BACKGROUND

Expert systems are a special type of computer system that utilizes a knowledge base to make specific decisions and provide guidance on a problem being analyzed. Medicine was one of the first areas where expert systems have been used. Clinical decision support systems (CDSS) have been developed to support clinicians in both diagnosis and treatment of patients. The functionalities of expert systems can be grouped into several categories, these are: diagnosis support, information retrieval, selection of appropriate drugs, image recognition, alerts, notifications planning and evaluation of therapeutic plans, including analysis of treatment plans for errors, omissions and inconsistencies based on the clinical data of the patient and the available knowledge base. Artificial intelligence and data mining techniques are commonly used in expert systems.

A more extensive discussion of issues related to such expert systems can be found in the publication by P. Jasłowska, M. Jasłowski, I. Jóźwiak “Zastosowanie systemów wspomagania decyzji klinicznych w diagnozowaniu chorób rzadkich”, Zeszyty Naukowe Politechniki Śląskiej, series: ORGANIZACJA I ZARZĄDZANIE, 2017, 102(1975): pp. 119-127; and in M. Alther, C. K. Reddy, “Clinical Decision Support Systems” in Healthcare Data Analytics, edited by C. K. Reddy, C. C. Aggarwal, Taylor&Francis Group 2015, each of which is incorporated herein by reference in its entirety.

A method and system for clinical decision support is known from the publication of European application EP3471106 A1, which corresponds to US Patent Publication 20190108917 which is incorporated herein by reference in its entirety. The document discloses a method for supporting clinical decisions for diagnosis or therapy of a patient using a medical imaging system, comprising the steps of: receiving a procedure order; based on the received procedure order, automatically identifying a clinical context of the ordered procedure; generating initial imaging data of at least a portion of the patient's anatomy; generating feature extraction data based on the identified clinical context and based on the initial imaging data; extracting at least one feature specific to the clinical context using the generated feature extraction data; describing at least one extracted feature specific to the clinical context to produce at least one extracted annotated feature; for an identified clinical context, selecting a similar case dataset from a reference database of case datasets based on at least one extracted annotated feature.

Magnetic resonance imaging, or MRI, is a non-invasive method of obtaining images of the interior of objects. It is used in medicine, where it is one of the basic techniques of imaging diagnostics (tomography), and in scientific research. Magnetic resonance imaging is based on the phenomenon of nuclear magnetic resonance.

One of the parameters obtained during magnetic resonance imaging is the nuclear relaxation times associated with the phenomenon of free precession. A distinction is made between longitudinal relaxation time T1, and transverse relaxation time T2. There are many publications indicating a variety of applications for relaxation time analysis. The most commonly studied tissues/systems are the nervous system, especially the brain, and connective tissues such as cartilage.

The techniques used for quantitative imaging vary considerably depending on the area of the body and the type of tissue being studied. Some studies have implemented relatively simple protocols using general purpose coils, while others have used complex and tailored approaches to image the heart or blood vessels. Some of the issues are discussed in more detail in the following entitled publications each of which is incorporated herein by reference in its entirety:
N. Schwendener, C. Jackowski, F. Schuster, A. Persson, M. J. Warntjes, and W.-D. Zech, “Temperature-corrected post-mortem 1.5 T MRI quantification of non-pathologic upper abdominal organs”, Int. J. Legal Med., vol. 131, No. 5, pp. 1369-1376, September 2017.
T. Rogers et al., “Standardization of T1 measurements with MOLLI in differentiation between health and disease—the ConSept study”, J. Cardiovasc. Magn. Reson., vol. 15, No. 1, p. 78, September 2013.
I. Mordi, D. Carrick, H. Bezerra, and N. Tzemos, “T1 and T2 mapping for early diagnosis of dilated non-ischaemic cardiomyopathy in middle-aged patients and differentiation from normal physiological adaptation”, Eur. Heart J. Cardiovasc. Imaging, vol. 17, No. 7, pp. 797-803, July 2016.
M. Granitz et al., “Comparison of native myocardial T1 and T2 mapping at 1.5 T and 3 T in healthy volunteers”, Wien. Klin. Wochenschr, 2018.
D. Mitsouras et al., “In vivo differentiation of two vessel wall layers in lower extremity peripheral vein bypass grafts: application of high-resolution inner-volume black blood 3D FSE”, Magn. Reson. Med., vol. 62, No. 3, pp. 607-615, September 2009.

There are many applications of (quantitative MRI) qMRI. These range from analysis of cartilage wear with age to assessment of pathological changes responsible for conditions such as Alzheimer's or Parkinson's disease. Relaxation time analysis is often used to visualize abnormalities occurring in the brain, and rather than being used as a direct measure of tissue status (healthy/abnormal), the imaging is used to calculate the volume of organ parts, which in neurology or cardiology are considered important indicators of health. Sometimes different sequences have shown different efficacy depending on the part of the organ imaged, as in the case of one brain study. Each of the following publications is incorporated herein by reference in its entirety.
E. Olsson et al., “Ultra-high field magnetic resonance imaging parameter mapping in the posterior horn of ex vivo human menisci”, Osteoarthr. Cartil., pp. 1-8, 2018.
R. Fermin-Delgado et al., “Involvement of globus pallidus and midbrain nuclei in pantothenate kinase-associated neurodegeneration: measurement of T2 and T2*time”, Clin. Neuroradiol., vol. 23, No. 1, pp. 11-15, March 2013.
C. Stehling et al., “Comparison of a T1-weighted inversion-recovery-, gradient-echo- and spin-echo sequence for imaging of the brain at 3.0 Tesla TT—Vergleich einer T1-gewichteten Inversion-Recovery-, Gradienten-Echo- and Spin-Echo-Sequenz zur zerebralen Bildgebung bei 3.0”, Rofo, vol. 177, No. 4, pp. 536-542, April 2005.

Most available solutions using magnetic resonance imaging data are designed to analyze specific tissues or systems working in conjunction with a specific magnetic resonance device. These tools often do not have the means to analyze other tissues, in particular the mammary gland tissue. The known and available tools do not sufficiently allow for the correct determination of relaxation times for this tissue.

Known solutions may therefore benefit from improvements.

### SUMMARY

In the case of the mammary gland cancer, diagnostic imaging capabilities are particularly important. Algorithms supporting advanced analysis of different types of images prove to be useful and clinically effective. The solution presented herein can not only improve or guide the diagnostic process, but also, for example, be used to monitor a patient's response to radiation or chemotherapy, or to assess the radicality of a surgical procedure.

The method for automatically determining a confidence interval based on quantitative magnetic resonance imaging (qMRI) data of various tissues is known from the publication of US application US2010225316 A1, the disclosure of which is incorporated herein by reference in its entirety. The method is based, on a relative evaluation of the results obtained by comparing a sample of one tissue with another, among other things, indicating relaxation times as indicators.

Proton relaxation times may be used in diagnostics. Calculations may be based on variations of the Bloch equations, published as early as 1946 and modified later to accommodate new measurement methods. Depending on the technology used, the recorded signal can take different forms and, in addition, a considerable number of factors can be taken into account that influence relaxation processes to a greater or lesser extent.

The radio pulse sequences used may manipulate the magnetisation of atoms in different ways, each of which can be targeted to achieve specific effects, such as enhancing the contrast of a given tissue, quenching it or minimizing magnetic field interference and the distortions caused by it. Depending on the procedure, the equipment used and the body area analyzed or the type of sample, examination times can range from a few minutes to several hours.

However, MRI, and in particular relaxation times, have not been used to analyze breast cancer lesions. In addition, when attempting to create possible standards of relaxation time values obtained, and to compare results between patients and separate studies, the difficulty is that tissues within the breast are characterised by considerable individual variability in terms of water content, fat content and overall structure. This variability is one of the difficulties not present to the same extent when imaging other areas of the body or tissues.

Known tools for the analysis of MRI results do not allow the analysis of relaxation times of any tissue. Based on the studies performed so far, it seems that the optimal approach is to use MRI in addition to mammography, because depending on the structure of the breast and the nature of potential neoplastic lesions not known in advance (e.g. during screening), either approach may be more effective.

When approaching the calculation of relaxation constants, factors that directly influence the value of the measured signal, such as the value of the external magnetic field or the use of contrast agents, and factors that additionally determine the way the signal is recorded, i.e. the measurement sequences used, should be taken into account.

The information about the applied sequence and the type of image (T1/T2) can be obtained directly from the data recorded by the scanner or from its analysis.

Several methods for recording relaxation times may be used. The earliest established standards include CP (Carra-Purcell method) or its modification the Carra-Purcell-Meiboom-Gill method for T2 images, both being modifications of the original Hahn spin echo method. Since their development, many new techniques have been developed to improve signal quality, reduce artifacts and shorten measurement times.

Caution should be exercised when comparing results of relaxation times measured with different sequences, which due to different SNR (signal to noise ratio) values and other not yet fully explained reasons, may introduce significant differences. There are also indications that the variability of the recorded signal depends on the type and parameters of the coils used in the resonance apparatus, as well as the applied deflection angle. However, this type of study has not been conducted on a large scale, involving a small number of biological and technical repetitions, and the greater reproducibility of the results obtained for phantoms than for patients suggests that a role for biological and anatomical factors cannot be excluded.

The value of the longitudinal T1 relaxation time of tissues changes depending on the external magnetic field—as the field strength increases, the T1 time increases. The transverse relaxation time T2 undergoes a similar phenomenon, although to a lesser extent, and there are also substances that react to changes in magnetic fields by shortening them, e.g. iron-based compounds. As devices operating in the fraction to a few Tesla range are commonly used, this presents an additional obstacle when comparing results from independently conducted studies. For example, ex vivo classification of breast cancer tissue at very low fields has been attempted. These attempts which were unsuccessful are described in Lee, S. J., Shim, J. H., Kim, K., Hwang, S. M., Yu, K. K., Lim, S., . . . & Kim, K. S. (2015), “Relaxation Measurement of Ex-Vivo Breast Cancer Tissues at Ultralow Magnetic Fields”, BioMed research international, 2015, which is incorporated herein by reference in its entirety.

Longitudinal relaxation is also highly dependent on the temperature of the sample, so that at least in exemplary arrangements, where the test is carried out in vitro and the tissue samples are cooled for transportation, it is important to keep their temperature constant during the test.

The exemplary arrangements descried herein solve the problem of determining relaxation times for histopathological samples of the mammary gland (in vitro) obtained from measurements with a magnetic resonance device, which may contain potential errors in the data sets that prevent the determination of correct relaxation times with commonly available tools. Exemplary arrangements provide a method for calculating relaxation times despite the presence of distorted or incomplete measurement series in the data set. Exemplary arrangements determine in a designated area of interest, parameters specific to the tissue under investigation, thus enabling the use of such determined parameters in an expert system.

The subject-matter of the exemplary method detects the presence of abnormal tissue by means of T1 and T2 relaxation times, wherein the T1 and T2 relaxation times are determined in the computing device on the basis of analysis of a data set obtained from a magnetic resonance apparatus from at least one tissue. The exemplary data set comprises data series which consist of scans corresponding to successive moments in time containing information about the intensity of the free induction decay signal, and is characterized in that it comprises the following steps:
(a) loading a data set from at least one tissue into the computing device, wherein the data set comprises at least one data series describing longitudinal relaxation and at least one data series describing transverse relaxation,
(b) determination of the region of interest (ROI), whereby the region of interest shall not change between successive scans in the data series,
(c) determination of the mean value of the free induction decay signal within the region of interest for each individual scan,
(d) detection of scans with outlier data in each data series, whereby the determined mean value of the intensity of the free induction fading signal within the region of interest for each scan in the data series is analyzed,
(e) if an outlier scan is detected, the determination and identification of the scan in the data series,
(f) determination of the relaxation time in the region of interest based on scans in the appropriate data series which are determined and flagged as having no outliers, with relaxation time T1 being determined from the longitudinal relaxation data series and relaxation time T2 being determined from the transverse relaxation data series, and
(g) classification of tissue as normal or abnormal on the basis of predefined values which are set according to the type of tissue to be assessed.

In exemplary arrangements, the isolation forest algorithm is used in step (d) of the method when detecting scans with outlier data. This algorithm was chosen because it is highly effective for low-surveyed data and works independently of its distribution. The results showed that it is suitable for solving the problem of determining relaxation times from perturbed or incomplete data.

In exemplary arrangements, the parameter of the algorithm, which is a coefficient describing the abnormality of a particular mean value in the analyzed data series, is no more than 0.2, and preferably no more than 0.1.

In exemplary arrangements, after step (e) of the region of interest (ROI) of the method, uncorrected relaxation times are determined based on all scans of the respective data series without excluding scans with outlier data, wherein the relaxation time T1 is determined from the data series describing longitudinal relaxation and the relaxation time T2 is determined from the data series describing transverse relaxation.

In exemplary arrangements, prior to step (d) detecting scans with outlier data is performed: verifying the times of echo (TE) and times of repetition (TR) recorded in the data series, wherein when the data series contains a constant time of echo (TE) and a variable time of repetition (TR) it is a valid data series describing longitudinal relaxation, and when the data series contains a constant time of repetition (TR) and a variable time of echo (TE) it is a valid data series describing transverse relaxation, and in case of different correlations the analysis is interrupted.

In exemplary arrangements, the determination of the relaxation time also includes:
based on the mean values of the free induction decay signal within the region of interest (ROI) determined in step (c), a characteristic of the variation of the free induction decay signal intensity over time is generated, where each time point corresponds to a separate scan, a relaxation curve is determined, which is an approximation curve corresponding to a predefined mathematical model, then
for the determined relaxation curve the relaxation time is determined, which is a parameter of this curve,
the fit measures of the individual models to the data are calculated,
the tissue is then classified as normal or abnormal on the basis of predefined values that are determined according to the type of tissue under investigation.

In exemplary arrangements, the determined relaxation times, relaxation curves, and characteristics of the variation of the intensity of the free induction decay signal over time are stored in the results database.

In exemplary arrangements, the predefined mathematical model of the relaxation curve is an exponential model, an exponential model with shift, or a biconvex model.

In exemplary arrangements, the tissue classification uses at least one of the following algorithms: naive Bayes classifier, neural network, support vector machine, random forest, and decision tree.

In exemplary arrangements, the at least one tissue to which the data set relates is a post-operative breast tumour sample, potentially cancerous.

An exemplary computer program product is characterised in that, when run on a computing device, it performs the steps of the method according to the exemplary methods. Such an exemplary computer program product includes a computer readable medium that includes non-transitory computer program instructions that when executed by one or more processors effectuate operations corresponding to at least some of the exemplary method steps.

The use of the exemplary methods in an expert system, which uses clinical data on a patient contained in a database to support diagnostic decisions, is characterised in that a predicted survival time is determined based on clinical data and classification results obtained by the method according to the exemplary arrangements.

A useful aspect of the exemplary arrangements is to provide objective data describing histopathological specimens, which together with other clinical data are an important element to support breast cancer diagnosis using an expert information system. The accepted relaxation time values and the initial classification of the sample are sent to the database. Both pieces of information may be used by the expert system to make a survival time prediction as well as other diagnostic determinations.

### BRIEF DESCRIPTIONS OF DRAWINGS

FIG. 1 is a schematic view of an exemplary magnetic resonance measuring system.

FIG. 2 is a schematic representation of the steps carried out in connection with an exemplary method.

FIG. 3A shows an exemplary relaxation curve without the removal of outlier data, including the calculated value of the mean square error of the fitted curve.

FIG. 3B shows the relaxation curve data of FIG. 3A after the removal of outlier data, and the calculated value of the mean square error of the fitted curve.

FIG. 4A illustrates a determined region of interest.

FIG. 4B shows a corresponding illustrative T1 map that corresponds to the region of interest in FIG. 4A.

FIG. 5 shows an example of fitting in exemplary relaxation curve according to different mathematical models and the determined values of relaxation time.

FIG. 6 is a table of terminology that includes terms in the Polish language that are utilized in the Figures, and their corresponding English terminology.

### DETAILED DESCRIPTION

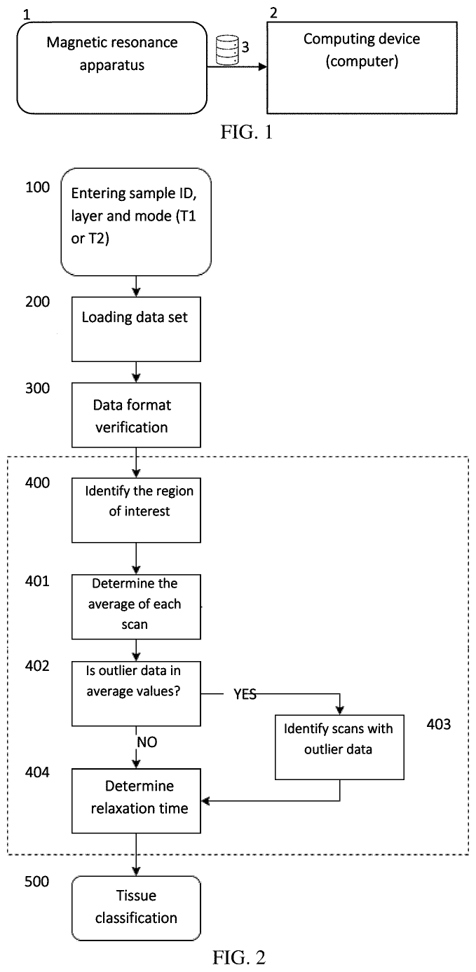

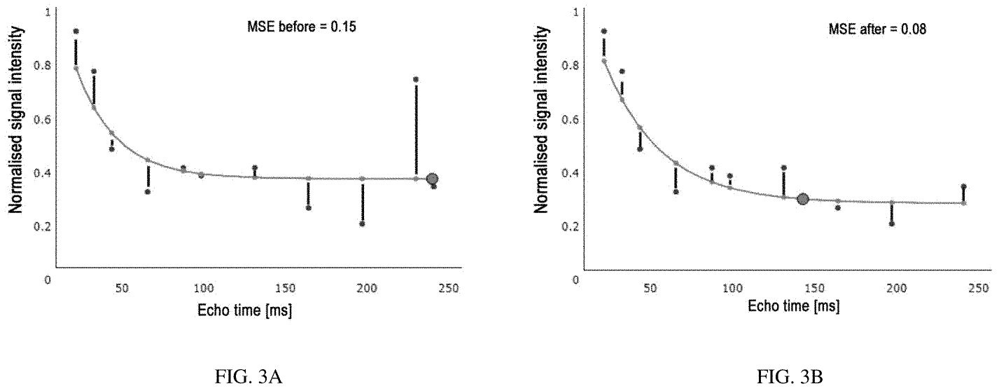

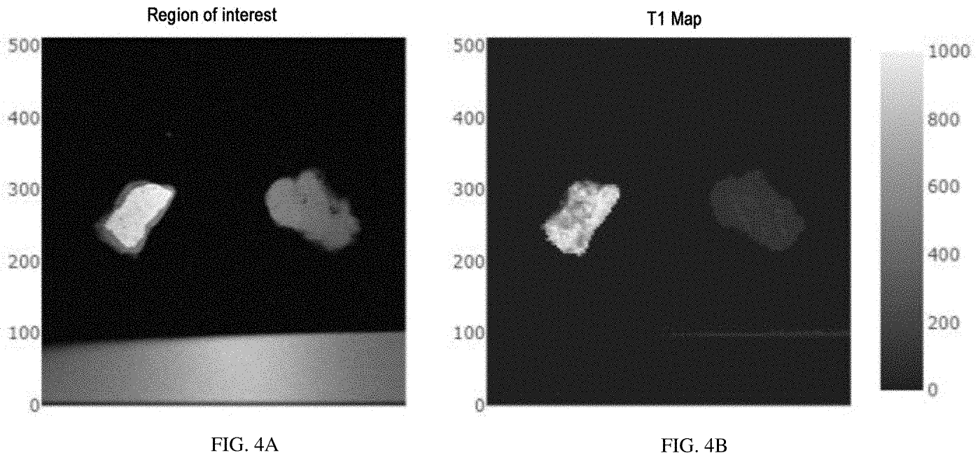

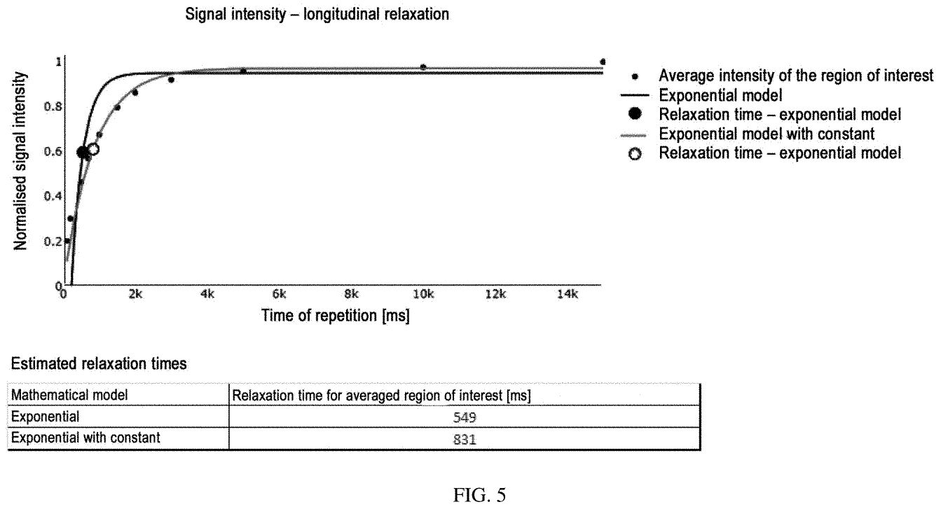

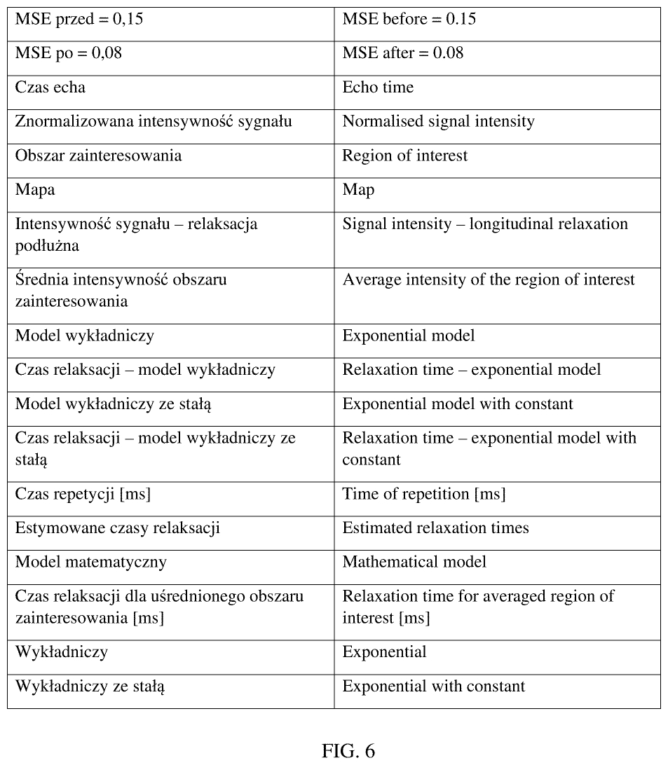

Referring now to the drawings and particularly to FIG. 1, there is shown therein a magnetic resonance apparatus generally indicated 1. The magnetic resonance apparatus is in operative connection with at least one computing device 2. The exemplary at least one computing device 2 is in operative connection with at least one data store 3 which includes at least one data set obtained through operation of the magnetic imaging apparatus from at least one tissue sample, which is alternatively referred to herein as a tissue.

In exemplary arrangements the at least one computing device 2 includes one or more circuits that are operative to communicate electrical signals. The one or more circuits include at least one processor and at least one data store. In exemplary arrangements the at least one processor may include a processor suitable for carrying out computer program instructions that are stored in the one or more associated data stores. The at least one processor includes or is in operative connection with a nonvolatile storage medium including instructions that include a basic input/output system (BIOS) or other interfaces. For example, processors may correspond to one or more of the combination of a CPU, FPGA, ASIC or any other integrated circuit or other type of circuit that is capable of processing data and program instructions. Exemplary data stores may correspond to one or more of volatile or nonvolatile memories such as random access memory, flash memory, magnetic memory, optical memory, solid-state memory or other mediums that are operative to store computer program instructions and data. Computer program instructions may include instructions in any of a plurality of programming languages and formats including, without limitation, routines, subroutines, programs, threads of execution, objects, methodologies, scripts and functions which carry out the actions such as those described herein. Structures for processors may include, corresponds to and utilize the principles described in the textbook entitled Microprocessor Architecture, Programming and Applications with the 8085 by Ramesh S. Gaonker, Sixth Edition (Penram International Publishing 2013) which is incorporated herein by reference in its entirety.

The exemplary data stores used in connection with exemplary arrangements may include one or more types of mediums suitable for storing non-transitory computer program instructions and data. Such mediums may include, for example, magnetic media, optical media, solid-state media or other types of media such as RAM, ROM, PROMs, flash memory, solid-state memory, computer hard drives or any other medium suitable for holding data and computer program instructions.

The method according to the exemplary arrangements and carried out with at least one computer device is shown schematically in FIG. 2. Detection of the presence of abnormal tissues in at least one tissue by means of relaxation times T1 and T2 includes making determinations through operation of the computing device 2 on the basis of analysis of a data set in the at least one data store 3 obtained from the magnetic resonance apparatus 1 from the at least one tissue. The data set comprises a series of data which corresponds to scans corresponding to successive moments in time and containing information about the intensity of the free induction decay signal.

The in vitro tissue samples analyzed in the exemplary arrangements are examined at a constant temperature, that is cooled to 5 degrees Celsius. The tissues are cooled for transport and their temperature is kept constant during testing. The examination of the tissues is carried out at a magnetic field strength of 1.5 T. The method in the exemplary arrangements includes the following steps:

100—entering the identifier for the tissue subject to analysis, layer and mode (T1 or T2). This information is stored in the at least one data store along with the associated data and the determined information,

200—loading a data set derived from at least one tissue into the computing device 2, wherein the data set comprises at least one data series describing longitudinal relaxation (T1) values and at least one data series describing transverse relaxation (T2) values,

400—determining an area of interest, wherein the area of interest (ROI) does not change between successive scans in each data series,

401—determining the average value of the free induction decay signal within the region of interest in each of the scans separately,

402—detecting scans with outlier data in each data series, wherein the determined average value of the intensity of the free induction fading signal within the region of interest for each scan in the data series is analyzed,

403—if a scan with outlier data is detected, determination of such a scan in the data series,

404—determining the relaxation time in the region of interest based on scans from the corresponding data series determined and flagged as non-outliers, with relaxation time T1 determined from the data series describing longitudinal relaxation, and relaxation time T2 determined from the data series describing transverse relaxation

500—classifying the tissue as normal or abnormal based on predefined values that are determined according to the type of tissue under investigation.

In the first aspect of this exemplary method, when detecting scans 402 with outlier data, an isolation forest algorithm is used for making the determination and the algorithm parameter contamination factor is 0.1.

In a second aspect of this exemplary method, after the step 403 in the region of interest (ROI), uncorrected relaxation times are determined based on all scans in the respective data series without excluding scans with outlier data, wherein the relaxation time T1 is determined based on the data series describing longitudinal relaxation and the relaxation time T2 is determined based on the data series describing transverse relaxation.

In a third aspect of this exemplary method, prior to the step of detecting scans 402 with outlier data, a verification of the times of echo (TE) and times of repetition (TR) recorded in the data series is performed, wherein when the data series includes a constant time of echo (TE) and a variable time of repetition (TR) it is determined to be a valid data series describing longitudinal relaxation, and when the data series includes a constant time of repetition (TR) and a variable time of echo (TE) it is determined to be a valid data series describing transverse relaxation. In case of different relationships, the data series is determined as not valid and the analysis is terminated.

In a fourth aspect of the exemplary method, determining the relaxation time 404 comprises:
based on the mean values of the free induction decay signal within the region of interest (ROI) determined in step (c), a characteristic of the changes in the intensity of the free induction decay signal over time is generated, where each time point corresponds to a separate scan, a relaxation curve is determined, which is an approximation curve corresponding to a predefined mathematical model, then
for the determined relaxation curve the relaxation time is determined, which is a parameter of this curve,
the fit measures of the individual models to the data are calculated,
the tissue is then classified as normal or abnormal on the basis of predefined values which are determined according to the type of tissue under investigation,
the determined relaxation times, relaxation curves, and characteristics of changes in the intensity of the free induction decay signal over time are stored in at least one data store in a results database.
In exemplary arrangements based on the measurements with a 95% probability (confidence level) of the values,
   T1 values are in the range 251.85 ms-301.94 ms
   T2 values are in the range of 114.19 ms-125.65 ms.

In the fifth aspect of the exemplary arrangement, the predefined mathematical model of the relaxation curve is an exponential model, an exponential model with shift, or a bi-exponential model, these models having different forms for longitudinal and transverse relaxation.

Based on the longitudinal relaxation process, the time T1 is determined. As the signal intensity increases over time, the T1 time is determined when the signal reaches 63% of its final value.

The basic exponential model used in an exemplary arrangement is based on Bloch's equations and the equation of the curve is as follows:

M=M₀[1−exp(−t/T₁)] (1)

In the above equation, M is the resultant value of the longitudinal magnetisation vector, M0 its final value (return to equilibrium after an earlier deflection by the radio signal), t is the time and T1 is the longitudinal relaxation time.

The shifted exponential model in an exemplary arrangement is a modification of the basic model by an additional factor k:

M=M₀[1−k(exp(−t/T₁))], (2)

For example k=2

M=M₀[1−2(exp(−t/T₁))] (3)

The bi-exponential model used in an exemplary arrangement in which the weights wₛ and w_{L} of the so-called short and long components of the relaxation time are defined, and T1s and T1L are the values of these two components. In this model the determined relaxation time consists of two numbers.

M=M₀wₛ[1−exp(−t/T₁ₛ)]+M₀w_{L}[1−exp(−t/T_{1L})] (4)

Transverse relaxation processes are used to calculate the T2 time. There are several fundamental difficulties associated with recording a fading signal. One of them is the need to compensate for the presence of a baseline or to deal with measurement noise. Although it is possible to find studies in which the performance of an exponential model has been found to be satisfactory, it is more common to find opinions that only more complex models are able to adequately represent the relaxation processes.

The basic exponential model describes the occurring phenomenon in some approximation:

M=M₀[exp(−t/T₂)] (5)

The shifted exponential model is much more faithful, and a fit with less error can be obtained by adding a constant b to the basic model. This is approximately equal to the baseline signal level (i.e. the minimum recorded after some time t>>T2):

M=M₀[exp(−t/T₂)]+b (6)

The bi-exponential model allows the relaxation curve to be fitted with even less error than previous methods. At the same time, it is more susceptible to distortions caused by the large scatter of the measured intensity values.

M=Mₛ[exp(−n(t/T₂ₛ))]+M_{L}[exp(−n(t/T_{2L}))]+s (7)

As for time T1 the relaxation time T2 determined in this model consists of two numbers.

In the sixth aspect of the exemplary arrangement of the tissue classification, the naive Bayes classifier algorithm and a random forest algorithm are used. The result is whether the sample described by the determined relaxation times is a normal tissue or a diseased tissue.

In a seventh aspect of the exemplary arrangement, the at least one tissue to which the data set relates is a post-operative breast tumour sample, potentially cancerous.

In a further aspect of the exemplary arrangement a computer program product comprises at least one medium bearing non-transitory computer program instructions and is characterized in that, when run on a computing device, it performs the steps of the method defined in the first arrangement and is therefore not repeated herein.

In a further aspect of the exemplary arrangement, the method according to the exemplary arrangement is applied to an expert system that uses clinical data on a patient contained in a database to support diagnostic decisions, characterised in that a predicted survival time is determined based on the clinical data and the classification results obtained according to the method.

The exemplary arrangements may also be used in diagnostic devices, providing a method for determining T1 and T2 relaxation times that is robust and tolerant to interference and missing data.

Thus, the exemplary arrangements achieve improved capabilities, eliminate difficulties encountered in the use of prior methods and approaches, and attain the useful results described herein.

In the foregoing description certain terms have been used for brevity, clarity and understanding. However, no unnecessary limitations are to be implied therefrom because such terms are used for descriptive purposes and are intended to be broadly construed. Moreover the descriptions and illustrations herein are by way of examples and the new and useful aspects of the arrangements are not limited only to the exact features that have been shown and described.

Having described the features, discoveries and principles of the exemplary arrangements, the manner in which they are utilized and operated, and the advantages and useful results attained, the new and useful features, methodologies, elements, arrangements, devices, parts, combinations, systems, operations, processes and relationships are set forth in the appended claims.

We claim:

1. A method for detecting a presence of abnormal tissue using T1 and T2 relaxation times, wherein the T1 and T2 relaxation times are determined in a computing device from analysis of a data set obtained through operation of a magnetic resonance apparatus on at least one tissue, wherein the data set comprises data series that includes scans corresponding to successive moments in time containing information about an intensity of a free induction decay signal, comprising:
(a) loading into the computing device the data set from the at least one tissue, wherein the data set includes at least one data series describing longitudinal relaxation and at least one data series describing transverse relaxation,
(b) determination of a region of interest (ROI), wherein the region of interest does not change between successive scans in each at least one data series,
(c) determining an average value of the free induction decay signal within the region of interest in each of the scans separately,
(d) detecting scans with outlier data in each data series, wherein detection of scans with outlier data is determined through analysis of the average value of the intensity of the free induction fading signal within the region of interest for each scan in the respective data series,
(e) responsive at least in part to detecting a scan with outlier data in (d), identifying the scan with outlier data in the at least one data series,
(f) determining the relaxation time in the region of interest based on scans in the corresponding at least one data series which have no outlier data, wherein relaxation time T1 is determined from the longitudinal relaxation data series and relaxation time T2 is determined from the transverse relaxation data series,
(g) classifying tissue as normal or abnormal on the basis of predefined values which are determined according to the type of tissue examined.

2. The method according to claim 1, wherein in step (d) the isolation forest algorithm is used for detecting scans with outlier data.

3. The method according to claim 1, wherein a parameter or an algorithm which is a coefficient describing an abnormality of a particular average value in the analyzed data series, is no more than 0.2, preferably no more than 0.1.

4. The method according to claim 1, wherein after step (e) in the region of interest (ROI), uncorrected relaxation times are determined based on all scans of the respective data series without excluding scans with outlier data, wherein relaxation time T1 is determined from the data series describing longitudinal relaxation and relaxation time T2 is determined from the data series describing transverse relaxation.

5. The method according to claim 1 and prior to (d), further comprising:
verification of the times of echo (TE) and times of repetition (TR) recorded in the data series, wherein when a data series contains a constant time of echo (TE) and a variable time of repetition (TR) a determination is made that the respective data series is a valid data series describing longitudinal relaxation, and wherein when a data series contains a constant time of repetition (TR) and a variable time of echo (TE) a determination is made that the respective data series is a valid data series describing transverse relaxation, and wherein in case of different correlations the analysis is interrupted.

6. The method according to claim 1 wherein the determination of the relaxation time in (f) comprises:
based on the mean values of the free induction decay signal within the region of interest (ROI) determined in (c), a characteristic of the changes in the intensity of the free induction decay signal over time is generated, where each time point corresponds to a separate scan, a relaxation curve is determined, being an approximation curve corresponding to a predefined mathematical model,
and further comprising:
for the determined relaxation curve, determination of the relaxation time, which is a parameter of the curve,
calculation of the mean square error as a measure of the fit of the individual models to the data, and
classification of the tissue as normal or abnormal on the basis of predefined values which are determined according to the type of tissue examined.

7. The method according to claim 6, wherein the determined relaxation times, relaxation curves, characteristics of changes in the intensity of the free induction decay signal over time, and measures of the fit of the individual models are stored in a results database.

8. The method according to claim 6, wherein the predefined mathematical model of the relaxation curve is an exponential model, an exponential model with a shift, or a bi-exponential model.

9. The method according to claim 1, wherein at least one of the following algorithms in (f) is used for tissue classification: naive Bayes classifier, neural network, support vector machine, random forest and decision tree.

10. The method according to claim 1, wherein at least one tissue to which the data set relates is a post-operative breast tumour sample, potentially cancerous.

11. The method according to claim 1, and further comprising:
subsequent to (g) providing the classification to an expert system that includes clinical data on a patient associated with the at least one tissue,
calculating a predicted survival time of the patient through operation of the expert system responsive at least in part to the classification and the clinical data.

12. At least one computer readable medium bearing non-transitory computer program instructions that when executed by a computing device are operative to configure the computing device to carry out the method steps recited in claim 1.
